(19) United States
(12) Patent Application Publication
(10) Pub. No.: US 2005/0159810 A1
(43) Pub. Date: Jul. 21, 2005

(54) Devices and methods for repairing cardiac valves

(76) Inventor: Farzan Filsoufi, New York, NY (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/760,151

(22) Filed: Jan. 15, 2004

(51) Int. Cl. A61F 2/24

(52) U.S. Cl. 623/2.1; 606/151; 623/2.36

(57) ABSTRACT

Devices and methods for the repair of a defective cardiac valve are provided. The implantable devices provide a leaflet coaptation surface and correct for one or more prolapsing segments of a valve leaflet. The methods involve implanting one or more devices within the defective cardiac valve. In certain embodiments, the devices and methods correct for billowing leaflets and/or a dilated valve annulus.

### FIELD OF THE INVENTION

The invention relates to devices and methods for facilitating and simplifying the repair of cardiac valves.

### BACKGROUND OF THE INVENTION

The human heart has four valves that control the direction of blood flow in the circulation. The aortic and mitral valves are part of the “left” heart and control the flow of oxygen-rich blood from the lungs to the body, while the pulmonic and tricuspid valves are part of the “right” heart and control the flow of oxygen-depleted blood from the body to the lungs. The aortic and pulmonic valves lie between a pumping chamber (ventricle) and major artery, preventing blood from leaking back into the ventricle after it has been ejected into the circulation. The mitral and tricuspid valves lie between a receiving chamber (atrium) and a ventricle preventing blood from leaking back into the atrium during ejection.

Various disease processes can impair the proper functioning of one or more of these valves. These include degenerative processes (e.g., Barlow's Disease, fibroelastic deficiency), inflammatory processes (e.g., Rheumatic Heart Disease) and infectious processes (e.g., endocarditis). In addition, damage to the ventricle from prior heart attacks (i.e., myocardial infarction secondary to coronary artery disease) or other heart diseases (e.g., cardiomyopathy) can distort the valve's geometry causing it to dysfunction.

Heart valves can malfunction in one of two ways. Valve stenosis is present when the valve does not open completely causing a relative obstruction to blood flow. Valve regurgitation is present when the valve does not close completely causing blood to leak back into the prior chamber. Both of these conditions increase the workload on the heart and are very serious conditions. If left untreated, they can lead to debilitating symptoms including congestive heart failure, permanent heart damage and ultimately death. Dysfunction of the left-sided valves—the aortic and mitral valves—is typically more serious since the left ventricle is the primary pumping chamber of the heart.

Dysfunctional valves can either be repaired, with preservation of the patient's own valve, or replaced with some type of mechanical or biologic valve substitute. Since all valve prostheses have some disadvantages (e.g., need for lifelong treatment with blood thinners, risk of clot formation and limited durability), valve repair, when possible, is usually preferable to replacement of the valve. Many dysfunctional valves, however, are diseased beyond the point of repair. In addition, valve repair is usually more technically demanding and only a minority of heart surgeons are capable of performing complex valve repairs. The appropriate treatment depends on the specific valve involved, the specific disease/dysfunction and the experience of the surgeon.

The aortic valve is more prone to stenosis, which typically results from buildup of calcified material on the valve leaflets and usually requires aortic valve replacement. Regurgitant aortic valves can sometimes be repaired but usually also need to be replaced. The pulmonic valve has a structure and function similar to that of the aortic valve. Dysfunction of the pulmonic valve, however, is much less common and is nearly always associated with complex congenital heart defects. Pulmonic valve replacement is occasionally performed in adults with longstanding congenital heart disease.

Mitral valve regurgitation is more common than mitral stenosis. Although mitral stenosis, which usually results from inflammation and fusion of the valve leaflets, can often be repaired by peeling the leaflets apart from each other (i.e., a commissurotomy), as with aortic stenosis, the valve is often heavily damaged and may require replacement. Mitral regurgitation, however, can nearly always be repaired but successful repair requires a thorough understanding of the anatomy and physiology of the valve, of the types of mitral valve dysfunction leading to mitral regurgitation and the specific diseases and lesions resulting in this dysfunction.

The normal mitral valve 2, as illustrated in FIGS. 1A and 1B, can be divided into three parts—an annulus 4, a pair of leaflets 6, 8 and a sub-valvular apparatus. The annulus 4 is a dense ring of fibrous tissue which lies at the juncture between the left atrium and the left ventricle. The annulus 4 is normally elliptical or more precisely “kidney-shaped” with a vertical (anteroposterior) diameter approximately three-fourths of the transverse diameter. The larger elliptical anterior leaflet 6 and the smaller, crescent-shaped posterior leaflet 8 attach to the annulus 4. Approximately three-fifths of the circumference of annulus 4 is attached to the posterior leaflet 8 and two-fifths of the annular circumference is attached to the anterior leaflet 6. The edge of each leaflet not attached to the annulus 4 is known as the free margin 10. When the valve is closed, the free margins of the two leaflets come together within the valve orifice forming an arc in the shape of a “smile” known as the line of coaptation 12. The corners of this “smile”, the two points on the annulus where the anterior and posterior leaflets meet (at approximately the 10 o'clock and 2 o'clock positions), are known as the commissures 14. The posterior leaflet 8 is usually separated into three distinct scallops by small clefts. The posterior scallops are referred to (from left to right) as P1 (the anterior scallop), P2 (the middle scallop) and P3 (posterior scallop). The corresponding segments of the anterior leaflet directly opposite P1, P2 and P3 are referred to as A1 (the anterior segment), A2 (the middle segment) and A3 (the posterior segment). The sub-valvular apparatus consists of two thumb-like muscular projections from the inner wall of the left ventricle (not shown) known as papillary muscles 16 and numerous chordae tendinae 18 (or simply “chords”) which are thin fibrous bundles which emanate from the tips of the papillary muscles 16 and attach to the free margin 10 or undersurface of the valve leaflets in a parachute-like configuration. The chords 18 are classified according to their site of attachment between the free margin 10 and the base of the leaflets. The marginal or primary chordae are attached at the free margin 10 of the leaflets and function to limit leaflet prolapse. The intermediate or secondary chordae are attached or attached to the underside of the leaflets at points between the free margin 10 and the base of the leaflets. The basal or tertiary chordae are attached to the base of the leaflets.

The normal mitral valve opens when the left ventricle relaxes (diastole) allowing blood from the left atrium to fill the decompressed left ventricle. When the left ventricle contracts (systole), the increase in pressure within the ventricle causes the valve to close, preventing blood from leaking into the left atrium and assuring that all of the blood leaving the left ventricle (the stroke volume) is ejected through the aortic valve into the aorta and to the body. Proper function of the valve is dependent on a complex interplay between the annulus, leaflets and subvalvular apparatus.

Lesions in any of these components can cause the valve to dysfunction, leading to mitral regurgitation—the regurgitation of blood from the left ventricle to the left atrium during systole. Physiologically, mitral regurgitation results in increased cardiac work since the energy consumed to pump some of the stroke volume of blood back into the left atrium is wasted. Overtime, the volume overload on the heart leads to myocardial remodeling in the form of left ventricular dilation and/or hypertophy. It also leads to increased pressures in the left atrium which results in the back up of fluid in the lungs and shortness of breath—a condition known as congestive heart failure.

Mitral valve dysfunction leading to mitral regurgitation can be classified into three types based on the motion of the leaflets (known as “Carpentier's Functional Classification”). Patient's with type I dysfunction have normal leaflet motion. Mitral regurgitation in these patients is due to perforation of the leaflet (usually from infection) or much more commonly due to distortion and dilatation of the annulus. Annular dilatation or distortion results in separation of the free margins of the two leaflets. This gap prevents the leaflets from coapting allowing blood to regurgitate back into the left atrium during systolic contraction.

Type II dysfunction results from leaflet prolapse. This occurs when a portion of the free margin of one or both leaflets is not properly supported by the subvalvular apparatus. During systolic contraction, the free margins of the involved portions of the leaflets prolapse above the plane of the annulus into the left atrium. This prevents leaflet coaptation and allows blood to regurgitate into the left atrium between the leaflets. The most common lesions resulting in Type II dysfunction include chordal or papillary muscle elongation or rupture due to degenerative changes (such as myxomatous pathology or “Barlow's Disease” and fibroelastic deficiency) or prior myocardial infarction.

Finally, Type III dysfunction results from restricted leaflet motion. Here, the free margins of portions of one or both leaflets are pulled below the plane of the annulus into the left ventricle. Leaflet motion which is restricted during both systole and diastole is evidence of a Type III A dysfunction. The restricted leaflet motion can be related to valvular or subvalvular pathology including leaflet thickening or retraction, chordal thickening, shortening or fusion and commissural fission, all of which may be associated with some degree of stenosis or fibrosis. Leaflet motion which is restricted during systole only is evidence of a Type III B dysfunction. Specifically, the leaflets are prevented from rising up to the plane of the annulus and coapting during systolic contraction. This type of dysfunction most commonly occurs when abnormal ventricular geometry or function, usually resulting from prior myocardial infarction (“ischemia”) or severe ventricular dilatation and dysfunction (“cardiomyopathy”), leads to papillary muscle displacement. The otherwise normal leaflets are pulled down into the ventricle and away from each other thereby preventing proper coaptation of the leaflets.

The anatomy and function of the tricuspid valve is similar to that of the mitral valve. It also has an annulus, chords and papillary muscles but has three leaflets (anterior, posterior and septal). The shape of the annulus is slightly different, more snail-shaped and slightly asymmetric. The demands on the tricuspid valve are significantly less than the mitral valve since the pressures in the right heart are normally only about 20% of the pressures in the left heart. Tricuspid stenosis is very rare in adults and usually results from very advanced rheumatic heart disease. Tricuspid regurgitation is much more common and can result from the same types of dysfunction (I, II, IIIA and IIIB) as the mitral valve. The vast majority of patients, however, have Type I dysfunction with annular dilatation preventing leaflet coaptation. This is usually secondary to left heart disease (valvular or ventricular) which can, over time, lead to increased pressures back stream in the pulmonary arteries, right ventricle and right atrium. The increased pressures in the right heart can lead to dilatation of the chambers and concomitant tricuspid annular dilatation.

The most common cause of insufficiency of the mitral valves in western countries is due to Type II dysfunction (leaflet prolapse). Repair of this dysfunction usually requires some type of leaflet resection and reconstruction along with, on occasion, additional leaflet and chordal procedures. The most common type of valve repair for Type II valve dysfunction is a quadrangular resection of the middle (P2) segment of the posterior leaflet. Resection of the P2 segment involves making perpendicular incisions from the free edge of the posterior leaflet toward the annulus, and then excising a quadrangular portion of the leaflet. Plication sutures are placed along the posterior annulus in the resected area and direct sutures are applied to the leaflet remnants to restore valve continuity. When excessive posterior leaflet tissue is present, such as in patients suffering from Barlow's disease, an ancillary procedure referred to as a sliding valvuloplasty is also performed. The P1 and P3 segments of the posterior leaflet are detached from the annulus and compression sutures are then placed in the posterior segment of the annulus. The gap between the two segments is then closed with interrupted sutures. As such, the height of the posterior leaflet is reduced to avoid postoperative systolic anterior motion (SAM). Sliding plasty is also indicated if a large quadrangle segment of the posterior leaflet is excised.

Many surgeons are comfortable repairing straightforward cases of P2 prolapse as described above. More complex Type II cases, including those with anterior leaflet involvement or prolapse at or near the commissures, usually require additional procedures such as chordal transfer, chordal transposition, placement of artificial chords, triangular resection of the anterior leaflet, sliding plasty or shortening of the papillary muscle and sliding plasty of the paracommissural area. Most surgeons, outside of specialized centers, rarely tackle these complex repairs and these patients usually receive a valve replacement.

In the early 1990s, Ottavio Alfieri popularized the concept of edge-to-edge repair, which was first described by Henry Nichols about 50 years ago. See Journal of Thoracic Surgery, Vol. 33, No. 1, January 1957. This repair technique consists of suturing together the edges of the leaflets at the site of regurgitation. This procedure can be applied at the paracommissural area (at the A1 and P1 segments of the leaflets) or at the middle of the valve (at the A2 and P2 segments; referred to as a “double orifice repair”). Initial studies showed a high rate of failure of the edge-to-edge repair particularly in patients with mitral regurgitation resulting from rheumatic fever and that a concomitant annuloplasty should be performed in every patient. More recently, the double orifice edge-to-edge technique has been applied to patients with Barlow's disease (typically involving prolapse of multiple segments) and bileaflet prolapse with satisfactory results. However, it has been found that the edge-to edge repair, particularly the double orifice technique, results in a significant decrease in mitral valve area which may result in mitral stenosis. Even without physiologic mitral stenosis, the decrease in orifice area increases flow velocities and turbulence, which can lead to fibrosis and calcification of the functioning valve segments. This will likely impact the long-term durability of this repair. Another factor, which may impact the long-term durability of the edge-to-edge technique, is the increased stress on the subvalvular apparatus of all segments. For example, in a patient with isolated A2 prolapse, suturing A2 to P2 increases the stress on the latter. In sum, current clinical data does not support the routine use of the edge-to-edge technique for the treatment of Type II mitral regurgitation.

Conventional procedures for replacing or repairing cardiac valves require the use of the heart-lung machine (cardiopulmonary bypass) and stopping the heart by clamping the ascending aorta (“cross-clamping”) and perfusing it with high-potassium solution (cardioplegic arrest). Although most patients tolerate limited periods of cardiopulmonary bypass and cardiac arrest well, these maneuvers are known to adversely affect all organ systems. The most common complications of cardiopulmonary bypass and cardiac arrest are stroke, myocardial “stunning” or damage, respiratory failure, kidney failure, bleeding and generalized inflammation. If severe, these complications can lead to permanent disability or death. The risk of these complications is directly related to the amount of time the patient is on the heart-lung machine (“pump time”) and the amount of time the heart is stopped (“cross-clamp time”). Although the safe windows for pump time and cross clamp time depend on individual patient characteristics (age, cardiac reserve, comorbid conditions, etc.), pump times over 4 hours and clamp times over 3 hours can be concerning even in young, relatively healthy patients. Complex valve repairs can push these time limits even in the most experienced hands. Even if he or she is fairly well versed in the principles of mitral valve repair, a less experienced surgeon is often reluctant to spend 3 hours trying to repair a valve since, if the repair is unsuccessful, he or she will have to spend up to an additional hour replacing the valve. Thus, time is a major factor in deterring surgeons from offering the benefits of valve repair over replacement to more patients. Devices and techniques which simplify and expedite valve repair would go a long way to eliminating this deterrent.

Within recent years, there has been a movement to perform many cardiac surgical procedures “minimally invasively” using smaller incisions and innovative cardiopulmonary bypass protocols. The purported benefits of these approaches include less pain, less trauma and more rapid recovery. This has included “off-pump coronary artery bypass” (OPCAB) surgery which is performed on a beating heart without the use of cardiopulmonary bypass and “minimally invasive direct coronary artery bypass” (MIDCAB) which is performed through a small thoracotomy incision. A variety of minimally invasive valve repair procedures have been developed whereby the procedure is performed through a small incision with or without videoscopic assistance and, more recently, robotic assistance. However the use of these minimally invasive procedures has been limited to a handful of surgeons at specialized centers in a very selected group of patients. Even in their hands, the most complex valve repairs cannot be performed since dexterity is limited and the whole procedure moves more slowly. Devices and techniques which simplify valve repair have the potential to greatly increase the use of minimally invasive techniques which would significantly benefit patients.

Thus, it is desirable to provide devices and procedures that overcome the shortcomings of the above-described valve repair procedures. It is desirable to provide a single device which, when operatively used, only requires a simplified procedure by which to repair a cardiac valve, and particularly to repair a mitral valve having Type II dysfunction. For example, it would be beneficial to provide a device which, when properly implanted corrects for leaflet prolapse thereby obviating the need to perform ancillary procedures to correct leaflet size and shape, to reattach or shorten chordae, etc. With such a device, most patients with Type II valve dysfunction could be corrected by device implantation alone. Simplifying the repair procedure would decrease the amount of time the patient's heart would need to be stopped and bypassed with a heart-lung machine and increase the likelihood that it could be performed minimally invasively. This would not only decrease the potential for complications, it would also allow a broader group of surgeons to perform the procedure.

### SUMMARY OF THE INVENTION

The present invention includes devices and methods of using the subject devices to repair cardiac valves. The present invention is particularly suitable for repairing regurgitant mitral and tricuspid valves having Type II valve dysfunction (leaflet prolapse).

An object of the present invention is to simplify repair procedures of a prolapsing leaflet and to obviate the need to perform any resection of the valve leaflets, chordal repair, transfer or shortening, or papillary repair or shortening, etc. Another object of the invention is to employ a single device and a single procedure to completely correct valve dysfunction due to leaflet prolapse. Proper implantation of the device in most cases obviates the need to perform chordal, papillary or other leaflet procedures as their collective ill-effects can be resolved solely by implantation of the subject device.

A feature of the present invention is the provision of an implantable device for facilitating proper leaflet coaptation without affecting the mobility of the leaflet and without reducing the effective valve area. The device is affixed to the affected leaflet at, over or under at least a portion of its prolapsing segment and provides a normalized coaptation surface area against which the opposing leaflet(s) may coapt. In certain embodiments, the device immobilizes or restrains the prolapsing portion or segment of the affected leaflet in order to permit leaflet coaptation during systole. By restraining the prolapsing segment and/or by providing an improved coaptation plane or surface, the devices facilitate coaptation of the leaflets(s) thereby eliminating the regurgitation.

### BRIEF DESCRIPTIONS OF THE DRAWINGS

FIG. 1A is a perspective view of a normal mitral valve having proper coaptation of the anterior and posterior leaflets.

FIG. 1B is a cross-sectional view of the left side of the heart illustrating the normal mitral valve of FIG. 1A.

FIG. 2A is a perspective view of a regurgitant mitral valve having a substantially prolapsing anterior leaflet.

FIG. 2B is a cross-sectional view of the left side of the heart illustrating the regurgitant mitral valve of FIG. 2A. The anterior leaflet of the mitral valve is shown prolapsing into the left atrium above the plane of the annulus as a result of a ruptured chord.

FIGS. 3A and 3B illustrate an embodiment of a device of the present invention for repairing a valve having a prolapsing leaflet.

FIGS. 4A and 4B illustrate another embodiment of a device of the present invention for repairing a valve having a prolapsing leaflet.

FIG. 5 illustrates yet another embodiment of a device of the present invention for repairing a valve having a prolapsing leaflet.

FIG. 6 is a cross-sectional view of the left side of the heart illustrating a regurgitant mitral valve having a prolapse smaller than that illustrated in FIG. 2A.

FIGS. 7A and 7B illustrate another embodiment of a device of the present invention for repairing a valve having a prolapsing leaflet.

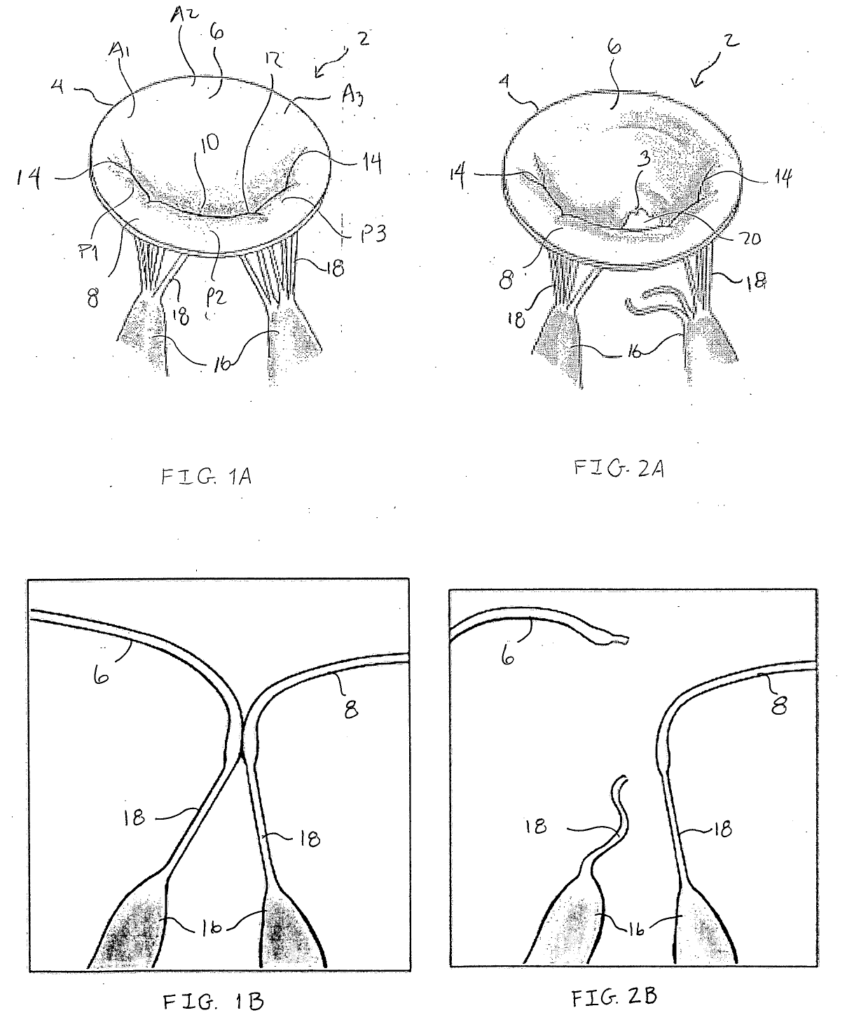

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided might be different from the actual publication dates which may need to be independently confirmed.

As mentioned above, the present invention is particularly suitable for repairing regurgitant mitral valves and particularly mitral valves having a Type II dysfunction. As such, the present invention is described in the context of mitral valves having Type II dysfunction; however, such application is exemplary only as the present invention is also suitable for the repair of tricuspid valves and other cardiac valves suffering from the same dysfunction or other dysfunctions.

Referring to the drawings, wherein like reference numbers refer to like components or anatomical structures throughout the drawings, FIG. 2A illustrates a top perspective view, i.e., as viewed from the left atrium, of a regurgitant mitral valve 2 having an annulus 4, anterior leaflet 6 and posterior leaflet 8. Mitral valve 2 has Type II valve dysfunction with substantial prolapse 3 of the A2 segment of the free margin of anterior leaflet 6 above the plane of the annulus 4 as a result of ruptured chordae 18. As better illustrated in FIG. 2B, the prolapse 3 prevents the anterior leaflet 6 from coapting with posterior leaflet 8 resulting in a gap 20 through which blood regurgitates from the left ventricle into the left atrium during systolic contraction.

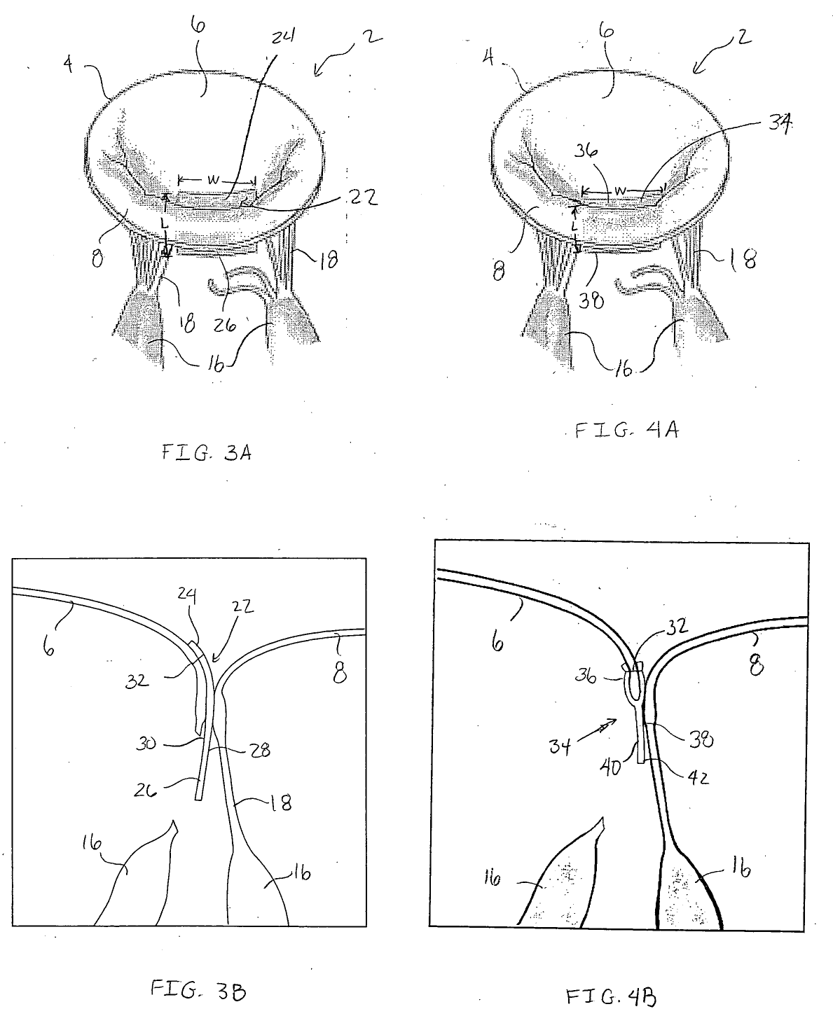

Various embodiments of a device of the present invention for repairing valve leaflet prolapse are illustrated in FIGS. 3, 4, 5 and 7, respectively. Each of the devices is made of an area or section of material, e.g., a strip, swatch, etc., configured for attachment to at least a portion of the prolapsing area of a valve leaflet, such as prolapsing anterior leaflet 6 of the defective mitral valve illustrated in FIGS. 2A and 2B. When operatively attached to the defective valve leaflet, the devices provide a prosthetic structure having a surface of coaptation against which an opposing leaflet, such as posterior leaflet 8, may coapt during systolic contraction of the heart and thereby ensure valve competency, i.e., close the gap caused by the prolapsing segment. More specifically, the device is affixed to the affected leaflet at, over or under at least a portion of its prolapsing segment and provides a normalized coaptation surface against which the opposing leaflet(s) may coapt. Unlike many prior art modalities of valve prolapse repair, the subject devices facilitate proper leaflet coaptation without affecting the mobility of the opposing leaflet (i.e., the leaflets are not connected together—unlike with edge-to-edge repair) and without reducing the effective valve area (i.e., the area of the valve orifice is maintained—unlike with edge-to-edge repair). In certain applications of the invention, the subject devices function to immobilize or restrain the prolapsing portion or segment of the affected leaflet. By restraining the prolapsing segment or by providing an improved or normalized coaptation plane or surface, the devices facilitate complete coaptation between the leaflets(s) thereby eliminating the regurgitation.

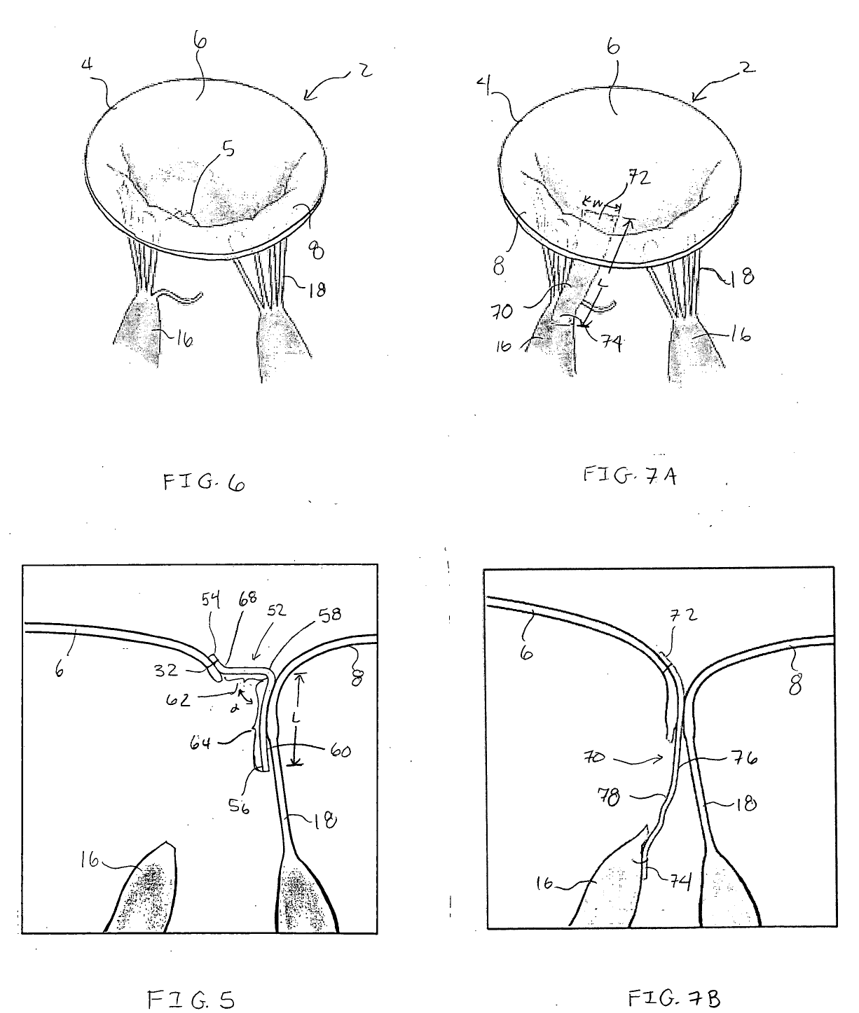

The subject devices may have any appropriate shape, surface area, thickness and cross-sectional profile necessary for the particular application, taking into consideration the length, height and surface area of the prolapsing leaflet segment and the thickness of the valve leaflet. While the illustrated embodiments are substantially square or rectangular in shape, they may have any other appropriate shape, including but not limited to elliptical, oval, triangular, etc. The devices have a width typically in the range from about 3 mm to about 30 mm, a length in the range from about 5 mm to about 40 mm, a thickness typically in the range from about 2 mm to about 10 mm and a coaptation surface area at least about 25 mm but may be larger or smaller depending on the application. The subject devices have a cross-sectional profile that may be substantially planar, slightly curved or bowed, or substantially curved where a curved configuration has at least one bend along its length. For curved profiles, the angle (see angle α in FIG. 5) formed thereby is typically in the range from about 75° to less than 180°, and more typically in the range from about 75° to less than 120°.

The prosthetic coaptation surface of the subject devices is configured to substantially anatomically mimic the surface of a normally functioning, natural valve leaflet, including but not limited to the texture and profile or curvature of the leaflet, so as to minimizing thrombotic effects on blood flow through the valve. As such, the coaptation surface is substantially smooth.

The devices are preferably made of a biologic or biocompatible material which may be rigid, semi-rigid, flexible, elastic or inelastic or a combination thereof. Additionally, the devices may be coated with a therapeutic-agent (e.g., anti-thrombogenic agent) for immediate or controlled, long-term release upon implantation.

The subject devices are further configured for attachment or affixation to the valve leaflet by any appropriate fixation means including but not limited to sutures, clips, fasteners, hooks, staples, biologic glue, etc.

While the aforementioned features are substantially shared by the various device embodiments of the present invention, certain other features may vary from embodiment to embodiment in order to accommodate various applications, valve types, the size and extent of prolapse and the physiological anomalies presented by the defective valve.

With certain embodiments, such as the embodiments of FIGS. 3A-3B, 4A-4B and 5, the distal end of the device is configured to override the free margin of the hosting leaflet, extending a distance beyond the free margin and into the ventricle upon coaptation of the leaflets without the need to affix or tether the distal end. As such, the devices function to extend the free margin of the treated leaflet. To this end, the leaflet extension devices are preferably made of a semi-rigid or rigid material, or a combination thereof, to provide a stable coaptation surface and to provide some stiffness to the device structure in order to withstand the pressures subjected to it by movement of the valve leaflets and the blood flow through the valve. If it is important to maintain or ensure a specific profile, e.g., curvature, of the device throughout its function, a rigid or semi-rigid material may further facilitate such. Rigid or semi-rigid materials suitable for use with the subject devices include, but are not limited to, metals (e.g., titanium), polymers (e.g., silicone, polyester and polytetrafluoroethylene (PTFE)), ceramics, carbon materials (e.g., graphite), Teflon, etc. The device structure may be solid, porous, have two or more interconnected parts or have a stent-like or woven structure reinforced with a material such as Dacron™ or PTFE.

For percutaneous applications, the subject devices may be made of material that allows them to be compressed to a low profile state for delivery through a catheter and subsequently expanded to an original sate upon deployment at the target implantation site. Suitable materials for percutaenous applications of the subject devices include but are not limited to shaped memory metal alloys (e.g., Nitinol) and silicone.

Additionally, the mass or weight of the devices may be selected to maintain the position of the device during normal valve function, to counter the force of the prolapsing segment during systole, as well as to obviate the need for tethering or fixing the distal end of the device to the valve or subvalvular structures. The weight of the subject device may also help to attenuate a billowing leaflet in which the body of the leaflet balloons into the left atrium above the plane of the annulus. Even leaflets which do not have a preexisting billowing problem may postoperatively develop such billowing after conventional mitral valve repairs as a result of increased chordal stress produced by the repair itself. The subject devices may further prevent such postoperative billowing. Suitable weights of the subject devices may range from about 5 mg to about 50 mg but may be heavier or lighter depending on the particular application.

Device 22 of FIGS. 3A and 3B includes a substantially planar area of material which has a square or rectangular shape or surface area; however, as mentioned above, any suitable shape may be employed. Device 22 has a slight curvature along its length L, a proximal or leaflet fixation end 24 and a distal or leaflet extension end 26. Leaflet fixation end 24 may have a thickness that tapers in order to ensure a flush surface with the natural leaflet surface to which it is attached. Unlike proximal end 24, distal end 26 is free or unattached when device 22 is operatively implanted. Device 22 further provides an outer or coaptation surface 28 and an under or inner surface 30, which may be slightly convex and concave, respectively. Outer or coaptation surface 28 preferably anatomically mimics the top or atrial surface of the hosting leaflet 6 in order to facilitate coaptation with the opposing leaflet 8. While device 22 is shown attached to the top or atrial surface of hosting leaflet 6, it may also be attached to the underside or ventricular surface of hosting leaflet 6.

Device 34 of FIGS. 4A and 4B has a similar shape and cross-sectional profile as device 22 of FIGS. 3A and 3B. Device 34 has an outer or coaptation surface 38, an under or inner surface 40, a proximal end 36 and a distal end 42. Device 34 differs from device 32 in that its proximal end 36 has a bifurcated configuration or a double layer configuration, as illustrated in FIG. 4B, designed to sandwich or hold the prolapsing free margin of a hosting leaflet there between.

As devices 22 and 34 primarily differ from each other in the construct of their respective proximal ends, the manner in which they engage with the hosting leaflet 6 also varies, as explained above. Nonetheless, similar fixation means 32 may be used to affix or adhere the devices to the hosting leaflet 6. Such means may include one or more of a plurality of mechanical fixation means, such as a suture, staple, clip, etc. Mechanical fixation means 32 is penetrated through the thickness of device 22 and into a least a portion of the thickness of the hosting leaflet. With device 34, fixation means 32 is penetrated through the first layer or segment, through the leaflet and into the second layer or segment. Such mechanical fixation means and the tools for applying them are known in the surgical arts. Alternatively, the fixation means may consist of a biologic glue. With device 22, the glue is applied to the proximal portion of the undersurface 26 of the device which is adhered to the top or atrial surface of the hosting leaflet. Alternatively, the glue is applied to the proximal portion of the top surface 28 of device 22 if the device is to be attached to the bottom or ventricular surface of the hosting leaflet. With a subvalvular attachment arrangement, it may be beneficial to ensure that the entire length of the prolapsing free margin is affixed to the device so as to provide a flush transition between the atrial leaflet surface and the outer or coaptation surface of the device. When using a glue to affix device 32, the glue is coated on the surfaces between the bifurcated portions of proximal end 36. Still yet, the devices may be ultrasonically welded to the surface of the hosting leaflet 6.

FIG. 5 illustrates another device 52 which functions and is affixed to a prolapsing leaflet similarly to the above-described devices; however, device 52 has at least one fairly pronounced curve or bend along its length so as to provide a “V” or “S” configuration. In the illustrated variation, device 52 has a bend 58 along its length thereby defining a proximal or horizontal portion 62 which terminates in a distal end 56, and further defining a distal, perpendicular or vertical portion 64. When operatively attached to a hosting leaflet 6, horizontal portion 62 extends beyond the free margin of the hosting leaflet 6 toward the opposing leaflet 8 substantially parallel to or within the same plane defined by the surface of the hosting leaflet 6. This extension 62 may help to compensate for a dilated or misshapen valve annulus (which results in a gaping valve orifice during systolic contraction of the heart). In other words, horizontal portion 62 bridges the residual gap between the leaflets caused by the dilated annulus and may obviate the need to use an annuloplasty ring. The length of horizontal portion 62 which extends beyond the free margin of hosting leaflet 6 is typically in the range from about 2 mm to about 15 mm, but may be shorter or longer depending on the extent of annular dilation. Between bend 58 and the proximal end 54, device 52 may have another, slighter bend or curve 68 in an opposite direction to bend 58 so as to deflect proximal end 54 for better anatomical placement on leaflet 6 (if the device is to be affixed to the atrial side of the leaflet). A leaflet coaptation surface 60 is defined substantially on the top surface of perpendicular portion 64 against which opposing leaflet 8 may coapt during systole. However, in operation, leaflet 8 may also coapt and contact bend 66 as well as the top surface of extension portion 62. Finally, proximal end 54 may be affixed by any means and in any manner as described above with respect to the other embodiments.

The length L (or the perpendicular portion of device 52) and width W of the above-described devices may depend on various factors including the width and height of the prolapsing portion of the leaflet. Generally, based on the typical surface area of the portion of a mitral valve leaflet affected by prolapse, the length L of the leaflet extension devices or horizontal extension portions thereof will range from about 5 mm to about 30 mm and the width W of the devices will range from about 5 mm to about 30 mm, but either dimension may be greater or smaller depending on the size of the prolapsing segment. Generally, the overall size of the device should be selected, and the device positioned, so as to overlap or cover at least about 50% of the surface area of the prolapsing segment. As mentioned above, it might be desirable to address a billowing portion of a leaflet under repair in addition to the prolapsing portion. As such, the device may be longer and/or wider (i.e., have an overall proximal surface area) to extend proximally and/or laterally (towards the annulus) over the atrial surface of the leaflet to restrain the billowing portion. The distance by which the free end of the devices extends within the ventricular space may depend on the extent of pre-operative prolapse, however, this extension distance typically ranges from about 10 mm to about 20 mm. The length, width and extension distance are preferably such that the devices of FIGS. 3, 4 and 5 do not contact surrounding anatomical structures, such as the papillary muscles 16 and the chordae 18, in order to minimize any inflammatory response or trauma.

It is preferable to minimize the contact area between the repair devices of the present invention and the leaflet surface. As such, the size or surface area of the device being used is preferably such that the areas of healthy or unaffected portions of the hosting leaflet are not in contact with the repair devices of the present invention. Accordingly, the smallest repair device possible should be used. However, the smaller the repair device (the lighter the mass), the greater the risk that it may not be able to withstand the blood pressure against it during systole and consequently be forced into the atrial chamber. Accordingly, it may be advantageous and/or necessary to further anchor a repair device at the implant location.

FIG. 6 illustrates a mitral valve 2 having a Type II valve dysfunction with a prolapse 5 of the A2 segment of the free margin of anterior leaflet 6 above the plane of the annulus 4 as a result of a ruptured chordae 18. This prolapse is far less pronounced than that illustrated in FIGS. 2A and 2B and, as such, requires less leaflet surface area to be immobilized.

The variation of the device of the present invention illustrated in FIGS. 7A and 7B may be suitable for smaller prolapses such as the one illustrated in FIG. 6. Device 70 is longer than the previously described embodiments, having both a proximal end 72 and a distal end 74 configured for fixation to the valve or subvalvular tissue structures. Specifically, unlike the previously described embodiments, the distal end 74 of device 70 extends further into the ventricle and is anchored to a subvalvular structure, such as papillary muscle 16 or the ventricle wall, and in essences, functions as an artificial chordae.

Device 70 must be sufficiently long and/or sufficiently flexible and/or elastic in order to accommodate the normal movement of the hosting leaflet, to minimize any unnecessary stress on the leaflet and/or to accommodate any residual prolapse of the leaflet. As such, a variety of natural and synthetic materials or combinations of natural and synthetic materials may be used. Suitable natural materials include but are not limited to human, bovine or porcine pericardial tissue. Suitable synthetic materials include but are not limited to super elastic metals (e.g., Nitinol), silicone, polyester and polytetrafluoroethylene (PTFE).

Again, while device 70 is shown having a rectangular configuration, any suitable shape may be employed. As with the previously described repair devices, device 70 is substantially planar and may be slightly curved, and has an outer or coaptation surface 76 and an under or inner surface 78. Outer surface 76 has a design which preferably anatomically mimics the top or atrial surface of the hosting leaflet 6 in order to facilitate coaptation with the opposing leaflet 8.

The length L and width W of device 70 depend on various factors including the width and height of the prolapsing portion of the leaflet (as well as the location of billowing if applicable), but also depends on the location at which distal end 74 is tethered. As such, the length L of device 70 typically ranges from about 20 mm to about 40 mm and the width W will range from about 3 mm to about 15 mm, but either dimension may be greater or smaller. It is important that the length of a subject device selected for a particular prolapse repair is not so short so as to restrict or restrain the leaflet and interfere with its normal function. Rather, it is far less detrimental to use a device having a length that is slightly longer wherein a slight prolapse of the leaflet remains, as the coaptation surface provided by implanted device compensates for such residual prolapse, i.e., the device provides sufficient surface area such that there is complete coaptation between the coaptation surface and the opposing leaflet during systolic contraction. The thickness of device 70 may be similar to that of the other devices discussed, and may taper at one or both ends for easier attachment to the leaflet 6 at the proximal end and to the selected anchoring site, e.g., papillary muscle 16, at distal end 74.

The fixation means mentioned above may also be used to affixed or adhered device 70 at its proximal end 72 to the hosting leaflet as well as its distal end 74 to the selected anchoring site. The means may be the same for all points of fixation or one type of fixation device may be employed to affix the proximal end and another may be used to attach the distal end.

While a number of exemplary embodiments of the devices of the present invention have been particularly described, those skilled in the art of cardiac valve surgery will appreciate that an unlimited number of device configurations is within the scope of the present invention. The suitability of a particular device configuration will depend on the particularities of the indication(s) being treated and the particular biases of the implanting surgeon. In other words, any suitable device shape, contouring, size, surface area and thickness may be employed having any suitable material. While the described devices are designed to treat a single prolapsing segment of a valve, other variations of the devices may address more than one prolapsing segment on the same leaflet.

Further, the present invention provides for systems which include at least one of the subject repair devices and fixation means, and may further include tools for applying the fixation means, catheters for delivering the repair devices in percutaneous approaches, and other ancillary tools necessary for implanting the subject devices.

The various methods of the present invention for using the subject devices and systems and for repairing cardiac valves will now be discussed in detail. As previously mentioned, the subject devices may be implanted using a surgical approach or a percutaneous approach. With either procedure, the prolapsing area of the subject valve is identified by preoperatively by gated MRI or echocardiography. From this assessment, a device is selected having the most appropriate configuration, size, shape and profile for optimum repair of the prolapsing segment.

With a surgical approach, an incision is made in the patient's chest. The conventional, and still most common, approach would be through a full median stemotomy. Other less invasive approaches include a partial stemotomy, a right (or less frequently left) full, partial or “mini” thoracotomy with video or robotic assistance, or port-access. Cardiopulmonary bypass is then established, typically by inserting cannulae into the superior and inferior vena cavae for venous drainage and into the ascending aorta for arterial perfusion. The cannulae are connected to a heart-lung machine which oxygenates the venous blood and pumps it into the arterial circulation. Additional catheters are usually inserted to deliver “cardioplegia” solution, which is infused into the heart after isolating it from the circulation with a clamp on the aorta and stop it from beating.

Once cardiopulmonary bypass and cardiac standstill have been achieved, the mitral valve is exposed by entering the left atrium and retracting the atrial tissue away using sutures or retraction devices. The atriotomy (entry incision) is usually made in the right side of the left atrium, anterior to the right pulmonary veins, although other approaches are occasionally used, especially in minimally invasive procedures. However, those skilled in the art will understand the necessary modifications to the procedure in order to access and repair the other cardiac valves through standard or less invasive approaches.

Once good exposure of the mitral valve has been achieved, the prolapsing area is confirmed by segmental valve anaylsis, i.e., each segment of each leaflet is carefully assessed using special forceps and hooks to determine its pliability, integrity and motion. Based on this assessment, the surgeon determines which segments require repair. One or more subject device is then operatively positioned at the prolapsing segment and the proximal end of the device is affixed to the leaflet. With the device embodiment of FIGS. 7A and 7B, the distal end is then affixed to a selected anchoring site, with the overall length of the device selected to ensure that the leaflet and chordae are not unduly stressed. Alternatively, the distal end may be affixed first followed by affixation of the proximal end to the leaflet.

Once the device or devices are secured, the repaired valve is tested to confirm a good line of coaptation between the leaflets without residual regurgitation. This is typically performed by injecting saline into the left ventricle until sufficient pressure develops to close the leaflets. Once the valve repair is complete, the atriotomy incisions are closed, the entrapped air is removed from the heart, the cross clamp is removed and the heart is reperfused causing it to start beating again. Soon there after the patient is gradually weaned off the support of the heart lung machine. The repaired valve is assessed using the transesophageal echocardiogram (TEE). If the repair is satisfactory, the cannulae are removed and the incisions are closed in a fashion consistent with other cardiac surgical procedures.

For percutaneous applications, valve repair devices made of a compressible-expandable material are preferably employed. The device is compressed to be received within a delivery catheter of appropriate length to reach the target valve via endovascular delivery. If treating the mitral valve, access to it may be made from various routes. If it is desirous to access the mitral valve by way of the left atrial chamber, delivery of the catheter is done through the venous system and then transatrially. For example, the catheter may be inserted into the femoral vein, translated through the inferior vena cava and into the right atrium. By means known by cardiac surgeons, the distal end of the catheter is made to cross the atrial septum into the left atrium. This approach may be preferable if attaching the repair device to the top or atrial surface of the targeted valve leaflet, but may also be used to attach the repair device to the bottom or ventricular surface. Alternatively, the mitral valve may be accessed by way of the left ventricle. For example, the catheter may be inserted into the femoral artery, translated through the aorta and made to cross the aortic valve into the left ventricle. This ventricular approach may be preferably if attaching the repair device to the bottom or ventricular surface of the targeted valve leaflet.

Regardless of the delivery route employed, once the catheter is positioned at the implant site, the selected repair device is advanced through the catheter and deployed at the mitral valve. The endovascular delivery procedure may be performed under echocardiographic or fluoroscopic guidance to help identify the best position for the repair device. Other tools such as a grasping device may be used to immobilize and hold the target leaflet while the repair device is positioned on and secured to it. The repaired valve is then assessed by TEE as described above. If residual regurgitation is detected, the position of the repair device may be adjusted.

With any type of repair approach, it may beneficial to use a means for temporarily attaching the device at the selected position on the leaflet in case adjustment is necessary after assessing the adequacy of the repair. To this end, if using sutures or fasteners, initially only a single stitch or fastener may be placed to secure the device. If TEE reveals that this initial position is not optimal, it will then be easier to remove just one stitch or fastener, thereby reducing damage to the leaflet tissue. It may be further advantageous to use releasable fasteners.

While the subject methods have been described in the context of implanting a single repair device, more than one of the subject devices may be employed, either on the same leaflet having more than one prolapsing section or on both leaflets (or three where applicable). Thus, the implant procedure may be repeated as necessary to address additional prolapsing segments on the same leaflet or on additional leaflets.

Also provided by the subject invention are kits for use in practicing the subject methods. The kits of the subject invention include at least one subject valve repair device of the present invention. Certain kits may include several subject devices having different sizes and/or shapes. Additionally, the kits many include certain accessories such as fixation means and devices for applying them as well as catheters for percutaneous implantation of the subject devices. Finally, the kits may include instructions for using the subject devices in the repair of cardiac valves. The instructions for use may include, for example, language instructing or suggesting to the user the most appropriate type or size of repair devices for treating a particular indication. These instructions may be present on one or more of the packaging, a label insert, or containers present in the kits, and the like.

It is evident from the above description that the features of the subject devices and methods overcome many of the disadvantages of prior art valve repair devices procedures including, but not limited to, minimizing the number or adjunctive procedures and instruments necessary to completely repair a cardiac valve, simplifying the repair procedure allowing more surgeons to offer this procedure to their patients and facilitating minimally invasive approaches to valve repair. As such, the subject invention represents a significant contribution to the field of cardiac valve repair.

While the present invention has been described with reference to the specific embodiments thereof, it should be understood by those skilled in the art that various changes may be made and equivalents may be substituted without departing from the true spirit and scope of the invention. In addition, many modifications may be made to adapt to a particular indication, material, and composition of matter, process, process step or steps, while achieving the objectives, spirit and scope of the present invention. All such modifications are intended to be within the scope of the claims appended hereto.

1. An implantable device for repairing a regurgitant cardiac valve having two or more leaflets and a subvalvular structure wherein at least one leaflet has a prolapsing segment, comprising:
a structure for attachment to the prolapsing leaflet, said structure defining a coaptation surface against which an opposing leaflet coapts during systolic contraction of the heart whereby the coaptation between the leaflets is normalized.

2. The device of claim 1 wherein said structure is rigid or semi-rigid.

3. The device of claim 1 wherein said structure is flexible.

4. The device of claim 1 wherein said structure is elastic.

5. The device of claim 1 wherein said structure has a proximal end configured for affixation to the prolapsing leaflet.

6. The device of claim 5 wherein said proximal end has a bifurcated configuration wherein a free margin of the prolapsing leaflet is positioned therein when said device is operatively affixed to the prolapsing leaflet.

7. The device of claim 1 wherein said structure has a distal end which extends freely beyond a free margin of the prolapsing leaflet when operatively implanted within the valve.

8. The device of claim 1 wherein said structure has a distal end configured for affixation to the subvalvular structure.

9. The device of claim 1 wherein said structure is substantially planar.

10. The device of claim 1 wherein said structure is curved or bowed.

11. The device of claim 10 wherein the curved structure defines an angle in the range from about 75° to less than 180°.

12. The device of claim 1 wherein said coaptation surface is configured to substantially mimic a normally function leaflet.

13. The device of claim 1 wherein said surface defines an area at least about 25 mm².

14. The device of claim 1 wherein said structure has a thickness in the range from about 2 mm to about 10 mm.

15. The device of claim 1 wherein said structure has a length in the range from about 5 mm to about 40 mm.

16. The device of claim 1 wherein the prolapsing leaflet also has a billowing section and wherein said surface has an area sufficient to immobilize the billowing section.

17. The device of claim 1 wherein the valve also has a dilated annulus resulting in a gap between the prolapsing leaflet and the opposing leaflet during systole and wherein a portion of said structure has a length sufficient to bridge the gap.

18. A system for repairing a regurgitant cardiac valve having two or more leaflets and a subvalvular structure wherein at least one leaflet has a prolapsing segment, comprising:
a structure configured for attachment to the prolapsing leaflet, said structure defining a coaptation surface against which an opposing leaflet coapts during systolic contraction of the heart wherein the coaptation between the leaflets is normalized;
a fixation means for attaching said structure to the prolapsing leaflet.

19. The system of claim 18 where said fixation means is selected from the group consisting of sutures, staples, clips, fasteners and glues.

20. A method for repairing a regurgitant cardiac valve having two or more leaflets and a subvalvular structure wherein at least one leaflet has at least one prolapsing segment, said method comprising the steps of:
providing a structure for attachment to the prolapsing leaflet, said structure defining a leaflet coaptation surface; and
implanting said structure at the regurgitant cardiac valve wherein, upon implantation, at least a portion of said structure extends between the two or more leaflets wherein a leaflet opposing said prolapsing leaflet coapts against said coaptation surface of said structure during systolic contraction of the heart whereby the coaptation between the two or more leaflets is normalized.

21. The method of claim 20 wherein said implanting comprises affixing said structure to said valve solely at said prolapsing leaflet.

22. The method of claim 21 wherein said structure is affixed to a top surface of said prolapsing leaflet thereby covering at least a portion of said top surface.

23. The method of claim 21 wherein said structure is affixed to an underside of said prolapsing leaflet.

24. The method of claim 21 wherein said affixing is accomplished by means of applying one or more selected from the group consisting of sutures, staples, clips, fasteners and glues.

25. The method of claim 21 wherein said affixing comprises affixing a proximal end of said structure to said prolapsing leaflet.

26. The method of claim 25 further comprising the step of affixing a distal end of said structure at a location on the subvalvular structure.

27. The method of claim 20 wherein said implanting comprises substantially immobilizing said at least one prolapsing segment.

28. The method of claim 20 further comprising maintaining a single orifice of said valve upon implanting said structure.

29. The method of claim 20 wherein the prolapsing leaflet has two prolapsing segments, said method further comprising implanting a second one of said structure in said valve.

30. The method of claim 29 wherein said first structure is affixed to the prolapsing leaflet at the first prolapsing segment and said second structure is affixed to the prolapsing leaflet at the second prolapsing segment.

31. The method of claim 20 wherein two of the leaflets have at least one prolapsing segment each, said method further comprising implanting a second one of said structure in said valve.

32. The method of claim 31 wherein said first structure is affixed to a first prolapsing leaflet and said second structure is affixed to a second prolapsing leaflet

33. The method of claim 20 wherein said implanting is performed percutaneously.

34. The method of claim 33 wherein said percutaneous implanting comprises using a catheter to deliver said structure to the valve to be repaired.

35. The method of claim 34 further comprising compressing said structure for delivery through said catheter.

36. The method of claim 35 further comprising expanding said structure upon delivery to the valve to be repaired.

37. The method of claim 20 wherein said cardiac valve is the mitral valve.

38. The method of claim 20 wherein the prolapsing leaflet also has a billowing section and wherein said implanting immobilizes the billowing section.

39. The method of claim 20 wherein the valve also has a dilated annulus resulting in a gap between the prolapsing leaflet and the opposing leaflet during systole and wherein said portion of said implanted structure extending between said leaflets bridges the gap.

40. The method of claim 20 wherein the structure contacts at least about 50% of the prolapsing segment.

41. A method for repairing a regurgitant cardiac valve having two or more leaflets and a subvalvular structure wherein at least one leaflet has at least one prolapsing segment, said method comprising the steps of:
providing a structure for attachment to the prolapsing leaflet, said structure defining a leaflet coaptation surface and an undersurface;
affixing said structure to the prolapsing leaflet wherein the undersurface of said structure overlies the prolapsing segment; and
extending at least a portion of said structure between the two or more leaflets wherein a leaflet opposing said prolapsing leaflet coapts against said coaptation surface of said structure during systolic contraction of the heart whereby the coaptation between the two or more leaflets is normalized.

42. The method of claim 41 further comprising affixing said structure at a location on the subvalvular structure.
